(19) United States
(12) Patent Application Publication
(10) Pub. No.: US 2023/0420103 A1
(43) Pub. Date: Dec. 28, 2023

(54) ACTIVITY MANAGEMENT APPLICATIONS AND SERVICES

(71) Applicant: Microsoft Technology Licensing, LLC, Redmond, WA (US)

(72) Inventors: Rui ZHU, Bellevue, WA (US); Alekhya NANDULA, Oakland, CA (US); Luke Nitish KUMAR, Vaughan (CA); Justin James WAGLE, Pacifica, CA (US)

(21) Appl. No.: 17/850,311

(22) Filed: Jun. 27, 2022

(51) Int. Cl. G16H 20/30; G06F 9/451

(52) U.S. Cl. CPC G16H 20/30; G06F 9/451

(57) ABSTRACT

Systems, methods, and software are disclosed herein that provide a computer-based user experience that allows a user to consume information about physical and digital activities undertaken by one or more users. In an implementation, a software application on a computing device communicates with an online service to obtain activity information indicative of such activities, as well as activity topics produced by the service. The application groups the activities into activity groups based at least on the topics produced for the activities, and displays the activity groups in a user interface to the application.

### TECHNICAL FIELD

Aspects of the disclosure are related to the field of computer software applications and services and, in particular, to activity management software and services.

### BACKGROUND

Activity management applications and services empower users to create healthy and productive habits with respect to online and offline computing experiences. Various solutions in the marketplace provide parental controls that allow parents to filter inappropriate websites, limit browsing to kid-friendly websites, set screen-time limits across devices, and otherwise place boundaries on a child's use of applications and devices. Similar features are also available in other contexts such as workplace and school environments.

One particular feature enjoyed by users of activity management applications is the ability to receive activity information on the activities undertaken by other people or even themselves. For example, a parent member of a family group can obtain activity information on each child in the group detailing the child's screen time and online usage. Indeed, a parent can receive a weekly email summarizing a child's activity in a concise and useful manner. Some activity management applications also provide location tracking capabilities allowing parents or others to find their loved ones, share their locations with others, and save frequently visited locations.

### Overview

Technology is disclosed herein that provides a computer-based user experience allowing a user to consume information about physical and digital activities undertaken by one or more users in a more insightful manner that provides greater value to the user. In various implementations, a software application on a computing device communicates with an online service to obtain activity information indicative of such activities (including both physical and digital activities). The activity information also includes activity topics produced by the service. The application groups the activities into activity groups, based at least on the topics produced for the activities, and displays the activity groups in a user interface to the application. Thus, an end-user can glean insights into activity topics of interest to another user derived from both physical and digital activities undertaken by the other user.

This Overview is provided to introduce a selection of concepts in a simplified form that are further described below in the Technical Disclosure. It may be understood that this Overview is not intended to identify key features or essential features of the claimed subject matter, nor is it intended to be used to limit the scope of the claimed subject matter.

### BRIEF DESCRIPTION OF THE DRAWINGS

Many aspects of the disclosure may be better understood with reference to the following drawings. The components in the drawings are not necessarily to scale, emphasis instead being placed upon clearly illustrating the principles of the present disclosure. Moreover, in the drawings, like reference numerals designate corresponding parts throughout the several views. While several embodiments are described in connection with these drawings, the disclosure is not limited to the embodiments disclosed herein. On the contrary, the intent is to cover all alternatives, modification's, and equivalents.

FIG. 1 illustrates an operating environment in an implementation.

FIG. 2 illustrates a topic process in an implementation.

FIG. 3 illustrates an activity process in an implementation.

FIG. 4 illustrates a service architecture in an implementation.

FIG. 5 illustrates an operational scenario in an implementation.

FIG. 6 illustrates a user experience in an implementation.

FIG. 7 illustrates a computing system suitable for implementing the various operational environments, architectures, processes, scenarios, and sequences discussed below with respect to the other Figures.

### DETAILED DESCRIPTION

Many activity management applications and services suffer from the common drawback of information overload. That is, users may be provided with so much activity information that it becomes difficult to glean useful insights from the information. Moreover, absent crises or other acute situations, much of the information provided by such applications and services goes unheeded or underutilized. Improvements to activity management applications and services are proposed herein to counter the trend of information overload by organizing and presenting activity information by topics that encompass digital and physical activities alike.

In various implementations, a software application on a computing device communicates with an online service to obtain activity information indicative of activities undertaken by a user. In addition, the activity information includes activity topics produced by the online service for the activities with respect to physical activities and digital activities. The application groups the activities into activity groups based at least on the topics produced for the activities and displays (or enables the display of) the activity groups in a user interface to the application. The user experience provided by the application allows a user to quickly and easily understand the general nature of the activities that another user has undertaken—or even activities undertaken by oneself.

In a brief example, an individual interacts with a user interface to an activity management application on a mobile device. The application presents the individual with a user experience that includes a screen detailing a list of one or more people associated with the individual's account. In some cases, the one or more people may include a group of people including the individual. In other cases, the group of people consists of only the individual. In still other cases, the group of people may not include the individual. In any case, the individual can navigate to another screen for any specific one of the people to view a summary of the person's activities, including activities for the individual, if so selected. The application obtains activity information for the selected person—which includes activity topics—and displays the activity topics in the screen view. The individual is able to see the activity topics without having to navigate and consume a lengthy and obscure list of activities.

In another brief example, a parent interacts with a user interface to an activity management application on a mobile device. The application presents the parent with a user experience that includes a screen detailing a list of children associated with the parent's account. The parent can navigate to another screen for any specific one of the children to view a summary of the child's activities. The application obtains activity information for the child—which includes activity topics—and displays the activity topics in the screen view. The parent is able to see the activity topics without having to navigate and consume a lengthy and obscure list of activities.

In some implementations, the application groups the activities based on a proximity of each of the activity topics to each other of the activity topics. The application may also identify a label for each one of the activity groups based at least on those of the activity topics in a given activity group. In the same or other implementations, the user interface may include multiple views of the activity groups filtered based on a characteristic of each of the activities. For example, the views could filter the activities based on recentness, frequency, distance, or the like. Each view may include a detailed view for each activity in an activity group. Example details include a description of the activity, a day of the activity, and a time of the activity.

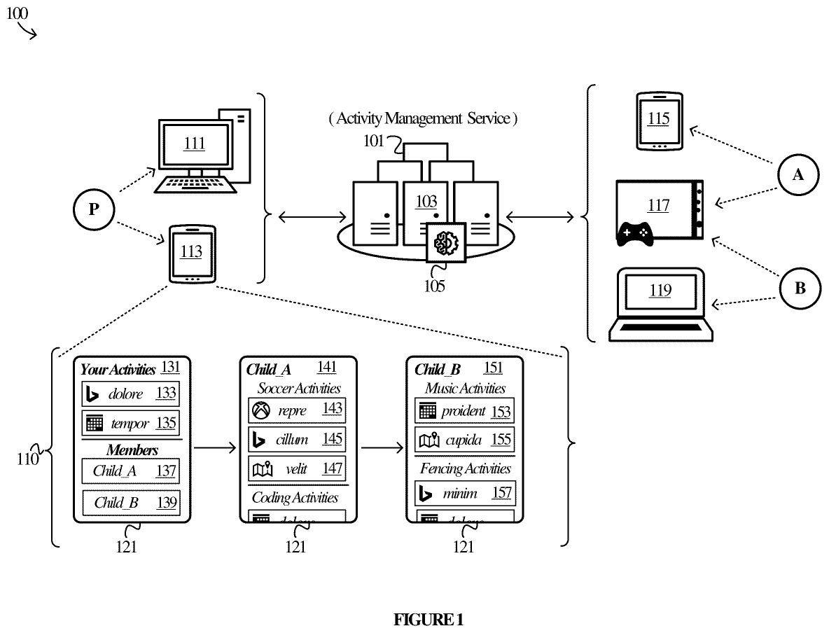

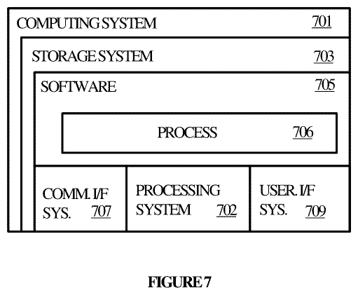

FIG. 1 illustrates an operational environment 100 in an implementation. Operational environment 100 includes activity management service 101, herein referred to as management service 101, as well as computing devices 111-119. Activity management service 101 employs one or more server computers 103 co-located or distributed across one or more data centers connected to computing devices 111-119. Examples of such servers include web servers, application servers, virtual or physical (bare metal) servers, or any combination or variation thereof, of which computing device 701 in FIG. 7 is broadly representative.

Computing devices 111-119 communicate with activity management service 101 via one or more internets and intranets, the Internet, wired and wireless networks, local area networks (LANs), wide area networks (WANs), and any other type of network or combination thereof. Examples of computing devices 111-119 include personal computers, tablet computers, mobile phones, gaming consoles, wearable devices, Internet of Things (IoT) devices, and any other suitable devices, of which computing device 701 in FIG. 7 is broadly representative.

Computing devices 111 and 113 each include one or more supervisory applications capable of providing a user experience with respect to activity management service 101. Examples include stand-alone applications that interface with activity management service 101 (e.g., the Family Safety app from Microsoft®), as well as features and functionality of other applications and/or operating systems that integrate a user experience with activity management service 101 into those contexts. Computing devices 115, 117, and 119 each include one or more agent applications capable of providing usage data and other telemetry information to activity management service 101. Such functionality may be provided as an integrated feature or service of any application or operating system, or as a stand-alone application or service dedicated to interfacing with activity management service 101.

Broadly speaking, the agent applications on computing devices 115, 117, and 119 detect and detail local application usage to activity management service 101 and enforce limits and other restrictions provided through activity management service 101. The supervisory applications on computing devices 111 and 113 provide visibility into the usage information communicated by the agent applications on computing devices 115, 117, and 119, while also providing controls for configuring the limits and restrictions enforced by the agent applications. For example, a supervisory application on computing device 113 allows a parent (user P) to oversee the usage of applications and services on computing devices 115, 117, and 119 by the parent's children (users A and B).

The agent applications send usage data to activity management service 101 indicative of a variety activities engaged in by users including: applications used; websites visited; search queries submitted; e-commerce sites visited; e-commerce searches; meeting details; device locations; games played; and peopled contacted, as well as numerous other activities. For example, an agent application may track and communicate browsing history including the browsing input (e.g., website address) and search terms used within the website.

In another example, the agent application may record and communicate search text including the keywords input to a search engine, results of the search, and results clicked on or otherwise selected by the user. Other examples include application information such as the name of a gaming property or service. Examples of location information include locations specified in meeting invites, locations of destinations visited by a user, locations of places searched, and the like, all of which may be captured and communicated by an agent application to an online service such as activity management service 101.

Activity management service 101 receives the usage data sent to it by the agent applications and provides it in various forms and formats to supervisory applications. For example, supervisory users are able to login to their management accounts to view the usage data for a given user in their group. This feature is especially useful for parents who wish to supervise their children in an easy and convenient manner. The supervisory user is not only able view the online activities of those under their purview but is also able to set limits and otherwise place boundaries on their online activity.

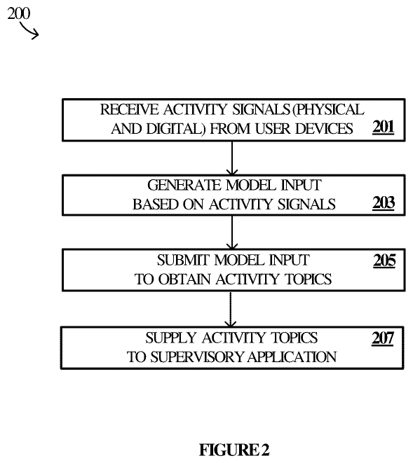

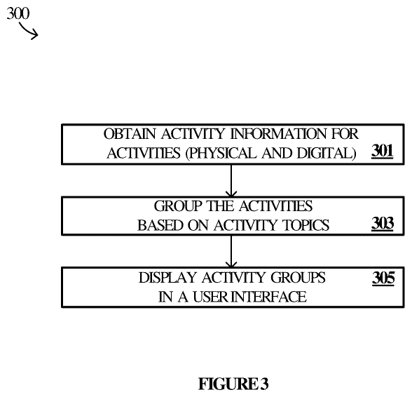

A technical improvement to such environments disclosed herein allows users in a supervisory position not only to monitor and control the online activity of their supervisees, but also to obtain topic-based insights about the supervisees that heretofore have not been available. In particular, FIG. 2 illustrates a process employed by activity management service 101 to collect activity signals and supply activity topics to supervisory applications, while FIG. 3 illustrates a corresponding process employed by the supervisory applications.

Process 200 in FIG. 2 illustrates a topic generation process in an implementation. Process 200 may be implemented in program instructions in the context of any of the software applications, modules, components, or other such elements of one or more computing devices that provide an activity management service (e.g., activity management service 101). The program instructions direct the computing device(s) to operate as follows, referring to a computing device in the singular for the sake of clarity.

In operation, a computing device employing process 200 receives activity signals from user devices (step 201). The activity signals include information indicative of digital and physical activities undertaken by users of the devices. A digital activity is an activity undertaken primarily with respect to an asset on a computing device such as playing a video game, searching an e-commerce website, placing calls, chatting online, or the like. A physical activity is an activity that is undertaken without regard for a computing device, but that may be memorialized in a digital record, examples of which include physical events attended by a user in-person memorialized in meeting invites, digital reservations, map locations (pins), and the like. Examples of physical events include sporting events, dining events, travel events (flights, car services, etc.), and any other type of in-person event that, other than it being memorialized electronically on a computing device, does not require a computing device to occur.

After receiving the activity signals, the computing device proceeds to generate input data based on the activity information, to be submitted to a topic model for evaluation (step 203). The model input data may be, for example, information extracted from the signals such as keywords used in a general search, keywords used in an e-commerce search, the name of a video game, location information, and the like. In the case of location information, a transform operation may be performed in some situations to transform non-textual information to a text format suitable for the topic model. For example, global positioning system (GPS) data in the form of latitude and longitude coordinates can be transformed to a text-based description of an area or region associated with the location.

The computing system submits the input data to a topic model to obtain activity topics (step 205). The output from the topic model includes text-based descriptions of topics learned from the input data. The input data may be supplied to the topic model on an individual, per-activity basis, such that the output represents a topic determined specifically for a single activity. The output data may be, for example, a single topic descriptor that fits the activity represented in the input data. However, the output data may also be multiple topic descriptors that fit the activity represented in the input data.

Alternatively, the input data may be supplied to the topic model in a batch-format such that multiple activities are represented in the input data. Under those circumstances, the topic model may output one or more topic descriptors that fit one, some, or all of the topics. For example, the output may be a single topic descriptor representative of a topic that fits all of the activities represented in the input data. In another case, the output may be multiple topic descriptors that fit all of the activities represented in the input data, such that each activity is described by multiple topic descriptors.

The computing system supplies the activity topics to a supervisory application on an end-user device (step 207). For example, the activity topics for a target user (e.g., a supervisee) may be sent from an activity management service to a supervisory application on a per-request basis, periodically, or at some other interval. In some cases, the activity topics may be stored temporarily for later access by a supervisory application. In any case, the supervisory application receives the activity topics and processes them in accordance with process 300, discussed below with respect to FIG. 3.

Process 300 in FIG. 3 illustrates a topic presentation process in an implementation. Process 300 may be implemented in program instructions in the context of any of the software applications, modules, components, or other such elements of one or more computing devices that provide an activity management application (or an activity management feature of an application or operating system). The program instructions direct the computing device(s) to operate as follows, referring to a computing device in the singular for the sake of clarity.

In operation, a computing device employing process 300 obtains activity information related to digital and physical activities undertaken by one or more users (step 301). For example, the computing device may receive activity information from an activity management service. The activity information comprises descriptions in a text format of the activities themselves, as well as topic descriptors for the activities. The topic descriptors may correspond to the activities on a one-to-one basis, meaning that each activity includes text indicative of the activity itself, as well as a topic descriptor for the activity.

Next, the computing device groups the activities based on the topic descriptors (step 303). In a simplified example, the activities may be sorted into groups such that activities having like topics are grouped with each other. In other cases, a k-nearest neighbor analysis of the topic descriptors may be performed to sort the activities into groups. In this manner, a text descriptor need not be an exact match with other descriptors in order for their corresponding activities to be grouped together.

Having sorted the activities into activity groups, the computing device displays the groups in a user interface (step 305). The activity groups may be displayed in the user interface in association with specific users. In addition, each activity group may be displayed with a label indicative of a topic determined for the activity group. As such, a user engaged with the user interface is able to quickly glean insights about the specific users by navigating to a view of each individual user's activity groups and reading or otherwise consuming the topics associated with the activity group.

Referring back to FIG. 1, the following describes an application of process 200 and process 300 with respect to the elements of operational environment 100, and a user experience 110 that results from their application. In operation, users A and B engage with computing devices 115, 117, and 119 in the course of a variety of digital and physical activities. The user may place calls and send messages, conduct Internet searches, shop online, attend in-person events, play video games, do homework, and otherwise undertake numerous activities. Here, it is assumed for exemplary purposes that user A has engaged with computing device 115 (e.g., a mobile phone) and computing device 117 (e.g., a gaming console), while user B has engaged with computing device 119 (e.g., a laptop computer) as well as computing device 117.

Agent applications on the computing devices track the activities undertaken by the users and send the activities to activity management service 101. The applications may report the activities in real-time (as they happen), in batch-mode, periodically, or at some other interval. The activities are communicated via update messages or other such mechanisms populated with information that describe details of the activities. The information may describe the name or identity of a property being used (e.g., a game title, website URL, application name), an identity of the user conducting the activity, a location of the activity, an identity of the device used for—or in association with—the activity, and a time and date of the activity.

The activity information may also include details of the activity, examples of which include the identity of other participants in the activity, meeting details of some activities (e.g., the location of a scheduled event and the identities of other people—if any), and content associated with the activity. For instance, a search activity can be identified in the activity information by the search engine used, the terms of the search, and results returned by the search. Further, the search activity could include subsequent activity associated with the search such as which results were clicked on or otherwise consumed by the user.

Location information is another example of activity details that may be provided in the activity information. For example, a scheduled event may be identified in the activity information by its time and date, subject, and location. In another example, the activity information may include the location of places visited by a user regardless of whether the occasions were planned, scheduled, or otherwise associated with a discrete calendar event. Location information may also be the subject of online searches such as those conducted via mapping applications or other search tools. Searches for physical locations (e.g., restaurants or convenience stores) may themselves be considered activities undertaken by a user which can be included in the activity information communicated to activity management service 101.

Activity management service 101 receives the activity information updates from the agent applications on computing devices 115-119 and proceeds to identify topics descriptors for the activities using topic engine 105. Topic engine 105 is representative of a system capable of taking activity information as input and identifying topics that fit the activities represented in the activity information. In some implementations, topic engine 105 may employ a machine learning model or other such algorithm(s) to identify the topics. As mentioned, activity management service 101 may convert some non-text formatted information to a textual format prior to submitting the activity information to topic engine 105. For instance, activity management service 101 may convert GPS data to textual descriptions of the locations that can be ingested by topic engine 105.

Topic engine 105 returns the topic descriptors to activity management service 101. In turn, activity management service 101 provides the topic descriptors to supervisory applications running on computing devices 111 and 113. It is assumed here for exemplary purposes that user P engages with the supervisory application on computing device 113 (e.g., a mobile phone) to learn more about the digital and physical activities of users A and B. Accordingly, the supervisory application directs computing device 113 to render a user interface 121 through which user P engages in user experience 110. It should be appreciated that the same or a similar user experience may be produced by the supervisory application running on computing device 111 (e.g., a desktop computer).

User experience 110 beings with a main screen 131 of the supervisory application displayed in user interface 121. The main screen 131 includes representations 133 and 134 of various activities undertaken by user P, who is logged into a supervisory account on the application. Representations 133 and 135 are each a graphical box, button, or other such user interface element that includes a brief description of an activity and potentially other helpful indicators, such as a graphic, associated with the activity. In this example, representation 133 is a graphical element associated with a search activity, while representation 135 is a graphical element associated with a calendar event. The user may touch, click-on, or otherwise select either representation in order to navigate into a more detailed view of each activity.

Main screen 131 also includes representations 137 and 139 of other users associated with the account including user A and user B. Representations 137 and 139 are each a graphical box, button, or other such user interface element that includes a brief description of a member associated with the supervisory account and whose activities are tracked by activity management service 101. It is assumed here for exemplary purposes that user P is the parent of user A and user B and as such, the users are represented as child-members of the account. In the supervisor/supervisee paradigm, user P is in the supervisory role, while users A and B are in supervisee roles. It may be appreciated that the supervisor/supervisee paradigm may be applicable to many other situations beyond just the parent-child relationship such as employer-employee relationships, and even non-hierarchical relationships where peers decide to enable the features and functionality described herein.

User P may touch, click-on, or otherwise select either one of the member representations in order to navigate to a member screen that displays the member's activities grouped by topic. Here, user P initially selects representation 137 to navigate to member screen 141. Member screen 141 includes a list of activities for user A (Child A) grouped by topic. The activities are displayed via representations 143-147, which are each a graphical box, button, or other such user interface element that includes a brief description of an activity and may include other indicators, such as a graphic, associated with the activity.

In this example, two activity groups are displayed in member screen 141 and labeled by topic: 1) soccer activities; and 2) coding activities. Other activity groups may be possible but would be shown in an offscreen portion of user interface 121 to which a user could scroll or otherwise navigate. The activity group labeled with the “soccer activities” topic includes representations of digital activities: representation 143 of a gaming activity, and representation 145 of a search activity. The soccer activity group also includes a representation 147 of a physical activity such as a soccer practice, game, or the like, attended by user A. Were the user to scroll down to the coding activities group, the group would include representations of both digital activities and physical activity(ies). The supervisory user is thus presented with a visualization of the activities undertaken by user A in a manner that provides topical insights into those activities.

Next, the supervisory user navigates to member screen 151 associated with user B. Member screen 151 includes a list of activities for user B (Child B) grouped by topic. The activities are displayed via representations 153-157, which are each a graphical box, button, or other such user interface element that includes a brief description of an activity and may include other indicators, such as a graphic, associated with the activity.

In this example, two activity groups are displayed in member screen 151 and labeled by topic: 1) music activities; and 2) fencing activities. Other activity groups may be possible but would be shown in an offscreen portion of user interface 121 to which a user could scroll or otherwise navigate. The activity group labeled with the “music activities” topic includes representations of digital and physical activities: representation 153 of a scheduled event, and representation 155 of a physical activity such as a recital or concert attended by user B. The fencing activities group includes a representation 157 of a search performed by the user with respect to fencing. The supervisory user is thus presented with a visualization that provides topical insights into the digital and physical activities of user B.

It may be appreciated that, in addition to the features and functionality discussed above, other capabilities may be provided, including the ability for users to delete activities from particular topic groups, as well as move activities to different topic groups. In addition, a system or sub-system on of computing device 113 or activity management service 101 may learn from and improve classifications based on such user feedback. For example, user actions such as deleting activities, moving activities, and renaming activities could be detailed and fed to a machine learning system that tailors the topic groups over time based on the feedback.

In another example, a system or sub-system on of computing device 113 or activity management service 101 may be capable of providing recommendations of additional physical and digital activities (e.g., notice of a new soccer field or a new web site resource). Other information may also be provided such as the time spent on an activity by a user. In some implementations, users may be able to share their activity data with other users. For example, individuals viewing their own activity topics may share one or more of the topics with friends, teammates, or the like.

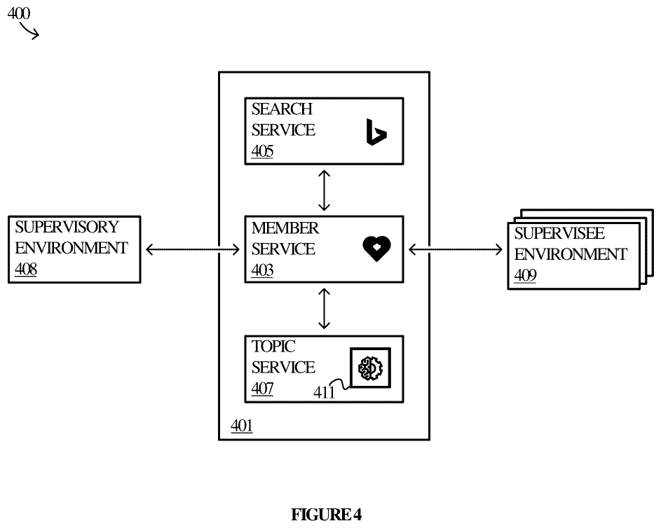

FIG. 4 illustrates an operational architecture 400 in an implementation. Operational architecture 400 is representative of an arrangement of software elements that may be employed to provide topical insights in the context of activity management applications and services. Operational architecture 400 includes a data center environment 401 which hosts member service 403, topic service 407, and search service 405. Data center environment 401 includes one or more physical server computers and other such equipment capable of hosting various services, of which computing system 701 is representative.

Member service 403 is representative of any software application(s) or component(s) capable of hosting an activity management service. Member service 403 communicates with search service 405, as well as topic service 407. Search service 405 is any service capable of taking search queries as input and providing search results as output. In particular, search service 405 is capable of taking location data in a numeric format as input and returning a location description in a text format. Topic service is any service capable of taking activity information as input and providing topic descriptors as output.

Member service 403 also communicates with supervisor environment 408 and supervisee environment 409. Member service receives activity information from supervisee environment 409, from which it produces activity groups and topic descriptors. The topic descriptors are provided by topic service 407. Member service 403 communicates the activity groups and labels to supervisory environment 408.

Supervisory environment 408 is representative of any software application or component capable of hosting a local activity management experience, examples of which include dedicated activity management applications, components or features of other applications, or combinations thereof. Supervisory environment 408 may be implemented in the context of any suitable computing device, of which computing device 701 is representative, such as laptop or desktop computers, mobile phones, table computers, and the like.

Supervisee environment 409 is also representative of any software application or component capable of hosting a local activity management experience, examples of which include dedicated activity management applications, components or features of other applications, or combinations thereof. Supervisee environment 409 may be implemented in the context of any suitable computing device, of which computing device 701 is representative, such as laptop or desktop computers, mobile phones, table computers, and the like.

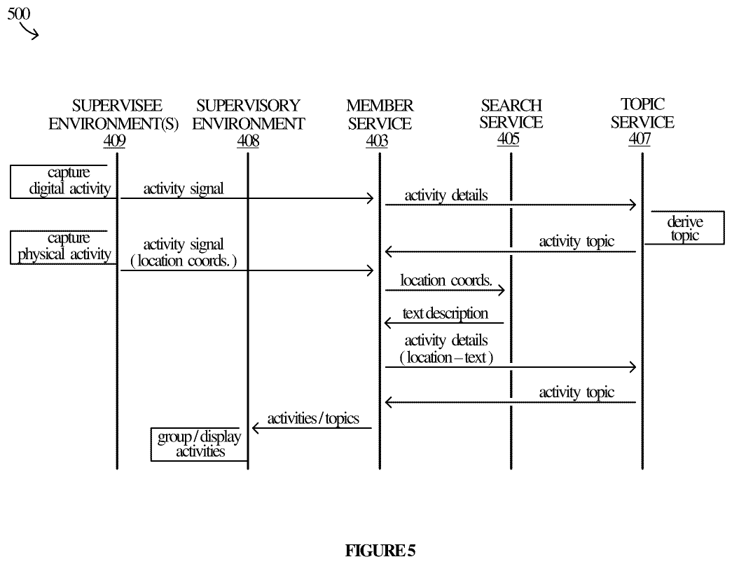

FIG. 5 illustrates an operational scenario 500 related to the elements of operational architecture 400. Operational scenario 500 illustrates an exemplary implementation of an activity management service with topical insights.

In operation, supervisee environment 409 captures and communicates digital activity undertaken by a user associated with the environment. Examples of such activity include online searches, e-commerce searches, people contacted, video games played, applications used, and other such activities relegated predominantly to an online experience. The format may be, for example, log messages communicated at the time of an activity that details a single activity, a single log message detailing multiple activities, or any suitable signaling format.

Member service 403 receives the signaling and communicates with topic service 407 to obtain topic descriptors for the indicated activities. For example, an online search for soccer equipment may be labeled with a “soccer” label, whereas an online search for sheet music may be labeled with a “music” label. However, the topic descriptors need not include words that overlap with the content of the activities themselves. For instance, the online search for soccer equipment could be labeled with an “outdoor sports” label. In some cases, multiple topics are returned for a single activity. In the preceding soccer example, the online search could be labeled with two topics: “soccer” and “outdoor sports.” The level of specificity or abstraction of a topic label may be configurable to fit various parameters such as a user preference, user preferences more generally, learned preferences, or the like.

In the meantime, it is assumed for exemplary purposes that the same user has undertaken a physical activity, a record of which is captured and communicated by supervisee environment 409. The activity may be, for example, a visit to a local sporting goods store, a visit to a soccer field, attendance at a soccer game, or some other similar event. However, the activity signaling may indicate no such details. Rather, the activity signaling (e.g., a log message) may indicate only (or predominantly) physical coordinates for the activity. Accordingly, member service 403 queries search service with the coordinates to obtain a text description of the location that can be used to obtain a topic descriptor.

Member service 403 queries topic service 407 with the text-based description of the location, and topic service 407 replies with a topic descriptor for the location. A topic descriptor for the location differs from a text-based description of the location in that it (the descriptor) describes a topic associated with the location, rather than its name. For example, whereas the text-based description of the location may identify “Contoso Field” as the name of the location, the topic descriptor would describe the location as associated with “soccer,” “outdoor sports,” or some other relevant topic.

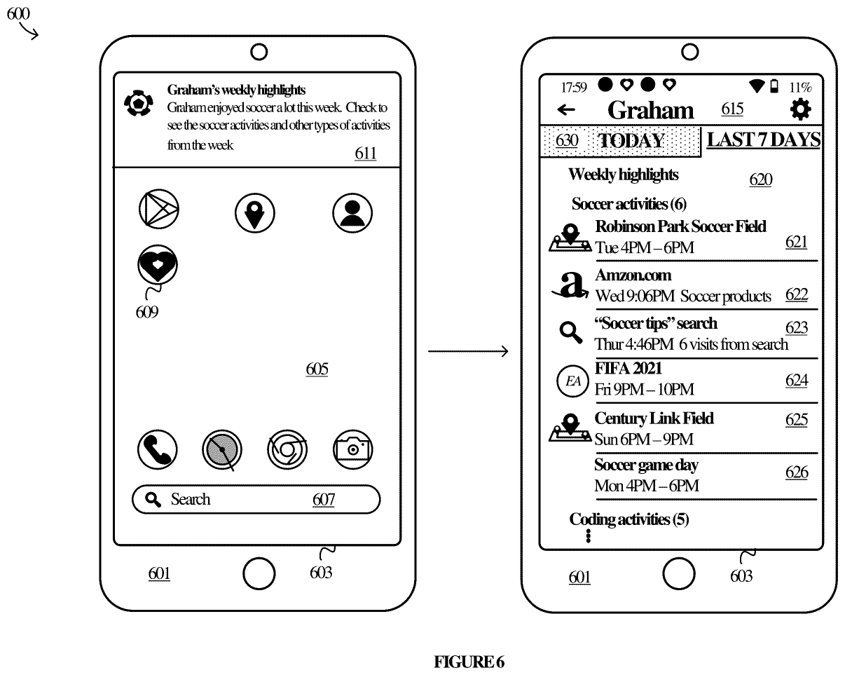

Member service 403 provides the activities and topics to supervisor environment 408 on-request, periodically, or at some other interval. Supervisory environment 408 receives the activities and associated topics and groups them into activity groups. The groups may then be displayed in a user interface to an application, examples of which are provided in FIG. 6.

FIG. 6 illustrates user experience 600 in an implementation. The user experience 600 involves a computing device 601 (e.g., a mobile phone) on which an activity management application provides user interface 603. Initially, user interface 603 displays a home screen view 605 on which various icons and other tools reside. For example, home screen view 605 includes a search bar 607 through which a user may make Internet searches, as well as an array of icons associated with various mobile apps. For instance, icon 609 is an icon for the activity management application.

A user can select icon 609 to launch the application. In addition, activity updates may also be received in the background and displayed in a notification bar 611. In this example, the notification bar 611 indicates an update related to a member associated with a user's account with the activity management service. The user can touch or otherwise select the notification bar 611 (or the icon 609) to launch the activity management application, which results in a screen 615 for the application coming into the foreground of user interface 603. It may be appreciated that screen 615 is one related to the person (Graham) identified in the update surfaced by notification bar.

Screen 615 is a screen produced by the application and includes multiple views selectable by the user to see member activities filtered based on various criteria. For instance, view 620 displays a view of member activities in the past seven days, whereas view 630 would display a view of member activities for the present day. Here, the activities relate to a single user—Graham—although it may be appreciated that others screens may display view of activities for multiple members.

In particular, view 620 illustrates activities grouped by two topics: 1) soccer, and 2) coding activities. The activities in the soccer group include activity elements 621-626. Activity element 621 represents a physical activity associated with soccer, as does activity element 625 and activity element 626. This is because the activities relate to physical locations that the member visited or intends to visit, as indicated by activity information recorded by a supervisory application on the member's device(s). In contrast, activity elements 622, 623, and 624 represent digital activities such as gaming, e-commerce searches, and general searches. For instance, activity element 622 relates to an e-commerce search, activity element 623 relates to a search engine query, and activity element 624 relates to a game played on a gaming console. Were the user to scroll down, digital and physical activities for the coding activities group would be visible. The supervisory user is thus able to quickly understand and appreciate at a glance that the supervisee user has undertaken activities related to soccer and coding, without having to navigate through and consume a potentially overwhelming list of activities.

Various technical effects may be appreciated with respect to the technology disclosed herein, including an improved ease of use, increased efficiency, and improvements in device performance. For example, the implementations disclosed herein allow a supervisory user to obtain a greater understanding of activities in an automatic, curated fashion through the visualization and organization of activity groups by topic. Such an arrangement makes the user experience more efficient, which also improves the efficiency and performance of the applications, devices, and services used to deliver such experiences. For example, a supervisory user need not scroll through or otherwise consume a long list of activities in order to gain insights about a supervisee's activities. Rather, the supervisory user is presented with the topical insights up-front, thereby eliminating the need for the time-consuming and processor intensive operations associated with inefficient experiences. The combination of digital and physical activities also provides a technical effect in that a supervisory user is presented with such information in one place, rather than having to jump between or amongst different activity lists or other such disorganized or lengthy visualizations.

FIG. 7 illustrates computing device 701 that is representative of any system or collection of systems in which the various processes, programs, services, and scenarios disclosed herein may be implemented. Examples of computing device 701 include, but are not limited to, desktop and laptop computers, tablet computers, mobile computers, mobile phones, and wearable devices. Examples may also include server computers, web servers, cloud computing platforms, and data center equipment, as well as any other type of physical or virtual server machine, container, and any variation or combination thereof.

Computing device 701 may be implemented as a single apparatus, system, or device or may be implemented in a distributed manner as multiple apparatuses, systems, or devices. Computing device 701 includes, but is not limited to, processing system 702, storage system 703, software 705, communication interface system 707, and user interface system 709 (optional). Processing system 702 is operatively coupled with storage system 703, communication interface system 707, and user interface system 709.

Processing system 702 loads and executes software 705 from storage system 703. Software 705 includes and implements activity management process 706, which is representative of activity management processes discussed with respect to the preceding Figures, such as process 200 and process 300. When executed by processing system 702, software 705 directs processing system 702 to operate as described herein for at least the various processes, operational scenarios, and sequences discussed in the foregoing implementations. Computing device 701 may optionally include additional devices, features, or functionality not discussed for purposes of brevity.

Referring still to FIG. 7, processing system 702 may comprise a micro-processor and other circuitry that retrieves and executes software 705 from storage system 703. Processing system 702 may be implemented within a single processing device but may also be distributed across multiple processing devices or sub-systems that cooperate in executing program instructions. Examples of processing system 702 include general purpose central processing units, graphical processing units, application specific processors, and logic devices, as well as any other type of processing device, combinations, or variations thereof.

Storage system 703 may comprise any computer readable storage media readable by processing system 702 and capable of storing software 705. Storage system 703 may include volatile and nonvolatile, removable and non-removable media implemented in any method or technology for storage of information, such as computer readable instructions, data structures, program modules, or other data. Examples of storage media include random access memory, read only memory, magnetic disks, optical disks, flash memory, virtual memory and non-virtual memory, magnetic cassettes, magnetic tape, magnetic disk storage or other magnetic storage devices, or any other suitable storage media. In no case is the computer readable storage media a propagated signal.

In addition to computer readable storage media, in some implementations storage system 703 may also include computer readable communication media over which at least some of software 705 may be communicated internally or externally. Storage system 703 may be implemented as a single storage device but may also be implemented across multiple storage devices or sub-systems co-located or distributed relative to each other. Storage system 703 may comprise additional elements, such as a controller, capable of communicating with processing system 702 or possibly other systems.

Software 705 (including activity management process 706) may be implemented in program instructions and among other functions may, when executed by processing system 702, direct processing system 702 to operate as described with respect to the various operational scenarios, sequences, and processes illustrated herein. For example, software 705 may include program instructions for implementing an activity management process as described herein.

In particular, the program instructions may include various components or modules that cooperate or otherwise interact to carry out the various processes and operational scenarios described herein. The various components or modules may be embodied in compiled or interpreted instructions, or in some other variation or combination of instructions. The various components or modules may be executed in a synchronous or asynchronous manner, serially or in parallel, in a single threaded environment or multi-threaded, or in accordance with any other suitable execution paradigm, variation, or combination thereof. Software 705 may include additional processes, programs, or components, such as operating system software, virtualization software, or other application software. Software 705 may also comprise firmware or some other form of machine-readable processing instructions executable by processing system 702.

In general, software 705 may, when loaded into processing system 702 and executed, transform a suitable apparatus, system, or device (of which computing device 701 is representative) overall from a general-purpose computing system into a special-purpose computing system customized to support activity management features, functionality, and user experiences. Indeed, encoding software 705 on storage system 703 may transform the physical structure of storage system 703. The specific transformation of the physical structure may depend on various factors in different implementations of this description. Examples of such factors may include, but are not limited to, the technology used to implement the storage media of storage system 703 and whether the computer-storage media are characterized as primary or secondary storage, as well as other factors.

For example, if the computer readable storage media are implemented as semiconductor-based memory, software 705 may transform the physical state of the semiconductor memory when the program instructions are encoded therein, such as by transforming the state of transistors, capacitors, or other discrete circuit elements constituting the semiconductor memory. A similar transformation may occur with respect to magnetic or optical media. Other transformations of physical media are possible without departing from the scope of the present description, with the foregoing examples provided only to facilitate the present discussion.

Communication interface system 707 may include communication connections and devices that allow for communication with other computing systems (not shown) over communication networks (not shown). Examples of connections and devices that together allow for inter-system communication may include network interface cards, antennas, power amplifiers, RF circuitry, transceivers, and other communication circuitry. The connections and devices may communicate over communication media to exchange communications with other computing systems or networks of systems, such as metal, glass, air, or any other suitable communication media. The aforementioned media, connections, and devices are well known and need not be discussed at length here.

Communication between computing device 701 and other computing systems (not shown), may occur over a communication network or networks and in accordance with various communication protocols, combinations of protocols, or variations thereof. Examples include intranets, internets, the Internet, local area networks, wide area networks, wireless networks, wired networks, virtual networks, software defined networks, data center buses and backplanes, or any other type of network, combination of network, or variation thereof. The aforementioned communication networks and protocols are well known and need not be discussed at length here.

As will be appreciated by one skilled in the art, aspects of the present invention may be embodied as a system, method or computer program product. Accordingly, aspects of the present invention may take the form of an entirely hardware embodiment, an entirely software embodiment (including firmware, resident software, micro-code, etc.) or an embodiment combining software and hardware aspects that may all generally be referred to herein as a “circuit,” “module” or “system.” Furthermore, aspects of the present invention may take the form of a computer program product embodied in one or more computer readable medium(s) having computer readable program code embodied thereon.

It may be appreciated that, while the inventive concepts disclosed herein are discussed in the context of activity management applications and services, they apply as well to other contexts such as productivity applications and services, gaming applications and services, virtual and augmented reality applications and services, business applications and services, and other types of software applications, services, and environments.

Indeed, the included descriptions and figures depict specific embodiments to teach those skilled in the art how to make and use the best mode. For the purpose of teaching inventive principles, some conventional aspects have been simplified or omitted. Those skilled in the art will appreciate variations from these embodiments that fall within the scope of the disclosure. Those skilled in the art will also appreciate that the features described above may be combined in various ways to form multiple embodiments. As a result, the invention is not limited to the specific embodiments described above, but only by the claims and their equivalents.

What is claimed is:

1. A computing apparatus comprising:
one or more computer readable storage media;
one or more processors operatively coupled with the one or more computer readable storage media; and
an application comprising program instructions stored on the one or more computer readable storage media that, when executed by the one or more processors, direct the computing apparatus to at least:
communicate with an online service to obtain activity information indicative of activities undertaken by a user and activity topics produced by the online service for the activities, wherein the activities include physical activities and digital activities;
group the activities into activity groups based at least on the topics produced for the activities; and
enable a display of the activity groups in a user interface to the application.

2. The computing apparatus of claim 1 wherein, to group the activities into the activity groups based at least on the activity topics, the program instructions direct the computing apparatus to group the activities based on a proximity of each of the activity topics to each other of the activity topics.

3. The computing apparatus of claim 2 wherein the program instructions further direct the computing apparatus to identify a label for each activity group, of the activity groups, based at least on those of the activity topics grouped into the activity group.

4. The computing apparatus of claim 1 wherein the user interface includes multiple views of the activity groups filtered based on a characteristic of each of the activities.

5. The computing apparatus of claim 4 wherein each view, of the multiple views, includes a detailed view for each activity in an activity group displayed in the view.

6. The computing apparatus of claim 5 wherein the detailed view includes a description of the activity, a day of the activity, and a time of the activity.

7. The computing apparatus of claim 6 wherein the activity group includes a physical activity and a digital activity.

8. The computing apparatus of claim 7 wherein the physical activity comprises an outdoor activity undertaken by the user at a physical location identified in the detailed view.

9. The computing apparatus of claim 8 wherein the digital activity comprises an online activity undertaken by the user with respect to an online service identified in the detailed view.

10. A method of operating a computing device, the method comprising:
communicating with an online service to obtain activity information indicative of activities undertaken by a user and activity topics produced by the online service for the activities, wherein the activities include physical activities and digital activities;
grouping the activities into activity groups based at least on the topics produced for the activities; and
displaying the activity groups in a user interface to an application running on the computing device.

11. The method of claim 10 wherein grouping the activities into the activity groups based at least on the activity topics comprises grouping the activities based on a proximity of each of the activity topics to each other of the activity topics.

12. The method of claim 11 further comprising identifying a label for each activity group, of the activity groups, based at least on those of the activity topics grouped into the activity group.

13. The method of claim 12 wherein the user interface includes multiple views of the activity groups filtered based on a characteristic of each of the activities.

14. The method of claim 13 wherein each view, of the multiple views, includes a detailed view for each activity in an activity group displayed in the view.

15. The method of claim 14 wherein the detailed view includes a description of the activity, a day of the activity, and a time of the activity.

16. The method of claim 15 wherein the activity group includes a physical activity and a digital activity.

17. The method of claim 16 wherein the physical activity comprises an outdoor activity undertaken by the user at a physical location identified in the detailed view.

18. The method of claim 17 wherein the digital activity comprises an online activity undertaken by the user with respect to an online service identified in the detailed view.

19. One or more computer readable storage media having program instructions stored thereon that, when executed by one or more processors, direct a computing apparatus to at least:
obtain activity information from an online service, wherein the activity information indicates activities undertaken by a user and activity topics produced by the online service for the activities, wherein the activities include physical activities and digital activities;
group the activities into activity groups based at least on the topics produced for the activities; and
enable a display of the activity groups in a user interface.

20. The computer readable storage media of claim 19 wherein method of claim 16 wherein the physical activities comprise an outdoor activity undertaken by the user, and wherein the digital activities comprise an online activity undertaken by the user.
